(19) United States
(12) Patent Application Publication
(10) Pub. No.: US 2025/0146998 A1
(43) Pub. Date: May 8, 2025

(54) MEASUREMENT METHOD FOR CHARACTERIZING FERRITE GRAIN SIZE AND PHASE FRACTION OF DUPLEX STAINLESS STEEL

(71) Applicant: Guilin University of Technology, Guilin (CN)

(72) Inventors: Jialong Shen, Guilin (CN); Jianing Zhao, Guilin (CN); Chen Zhang, Guilin (CN); Xi Liu, Guilin (CN); Yufeng Chen, Guilin (CN); Zhengbing Meng, Guilin (CN); Yibing Li, Guilin (CN)

(21) Appl. No.: 18/791,281

(22) Filed: Jul. 31, 2024

(30) Foreign Application Priority Data
Nov. 7, 2023 (CN) 202311468359.7

(51) Int. Cl. G01N 33/204; G01N 27/02

(52) U.S. Cl. CPC G01N 33/204; G01N 27/025

(57) ABSTRACT

Disclosed is a measurement method for characterizing ferrite grain size and phase fraction of duplex stainless steel, including the steps of: conducting heat treatment on industrial pure iron and duplex stainless steel respectively; conducting wire cutting on the samples to obtain the samples to be tested; measuring ferrite grain diameters and phase fractions of the samples to be tested; testing the samples to be tested based on an improved U-shaped sensor probe to obtain test data; constructing a response relational database; obtaining the information of the sample of arbitrary ferrite grain size and phase fraction. The present invention adopts an innovative electromagnetic characterization method to measure single microcomponents in the steel and iron materials, which can be applied to the measurement of smaller samples and improve the measurement efficiency.

### CROSS-REFERENCE TO RELATED APPLICATIONS

This application claims priority of Chinese Patent Application No. 202311468359.7, filed on Nov. 7, 2023, the entire contents of which are incorporated herein by reference.

### TECHNICAL FIELD

The present invention belongs to the technical field of application of electromagnetic non-destructive testing, in particular to a measurement method for characterizing ferrite grain size and phase fraction of duplex stainless steel.

### BACKGROUND

The performance of steel and iron materials is closely related to their microstructure, and the mechanical performance of the materials can be greatly improved by controlling the microstructure of materials online in a production process. The premise of effective control of the microstructure is to accurately characterize the microstructure. How to accurately and quickly characterize the microstructure (grain size, phase composition, phase fraction, phase distribution and morphology) of the steel and iron materials is the key link to realize online quantitative control of the microstructure.

The existing microstructure characterization technology mainly uses indirect characterization such as metallographic observation, mathematical model calculation or online temperature measurement by partial sampling, which is easily affected by the production environment or model accuracy, and cannot dynamically adjust the production system in real time according to feedback results. The frequently-used ultrasonic testing mainly uses the propagation and reflection characteristics of ultrasonic waves in substances, and evaluates the internal structure, defects and performance of a measured object by detection of the propagation time of the wave and reflecting signals. Radiographic testing uses the penetration of rays and the absorption or scattering characteristics of a tested substance for the rays to evaluate the internal structure, defects or material properties of the object. These nondestructive testing technologies are limited by that their testing principles cannot characterize the internal microstructure of materials or production and installation environments cannot satisfy testing requirements, and are difficult to accurately characterize the microstructure of steel and iron online.

A multi-frequency electromagnetic nondestructive testing probe can test small changes in the electromagnetic properties of the materials caused by changes in the microstructure of the materials. Therefore, the use of the multi-frequency electromagnetic nondestructive testing technology to characterize the evolution process of the microstructure online has obtained certain scientific research and preliminary industrial application. The main principle is to evaluate the microstructure characteristics of steel and iron by measuring the response of steel and iron materials to electromagnetic waves of different frequencies, and changes in magnetic saturation effect, magnetic hysteresis effect and magnetic permeability. Nowadays, multi-frequency electromagnetic characterization technologies are mainly applied to the superposed/cooperative characterization of multiple microcomponents. However, the research on the characterization of a single important component in the multi-microcomponent structure has not been reported, and the obtained electromagnetic signal cannot be associated with an important single microcomponent. Therefore, it is particularly important to quantitatively analyze the influence features of each single microcomponent on the electromagnetic properties of steel and iron and further clarify the relationship between microcomponents and electromagnetic response.

### SUMMARY

The purpose of the present invention is to provide a measurement method for characterizing ferrite grain size and phase fraction of duplex stainless steel, so as to solve the problems existing in the prior art.

To achieve the above purpose, the present invention proposes a measurement method for characterizing ferrite grain size and phase fraction of duplex stainless steel, comprising the following steps:
setting heat preservation and cooling conditions respectively, and conducting heat treatment on industrial pure iron and duplex stainless steel respectively to obtain samples with different grain sizes and different phase fractions;
conducting wire cutting on the samples after heat treatment to obtain the samples to be tested;
measuring ferrite grain diameters and phase fractions of the samples to be tested to obtain measurement results;
testing the samples to be tested based on an improved U-shaped sensor probe to obtain test data;
constructing a frequency inductance curve based on the test data and the measurement results; and constructing a response relational database based on the frequency inductance curve;
obtaining the information of the sample of arbitrary ferrite grain size and phase fraction based on the response relational database.

Optionally, the samples to be tested comprise metallographic observation samples with the size of 10×10×5 mm³ and electromagnetic testing samples with the size of 55×10×5 mm³.

Optionally, the process of measuring the ferrite grain diameters of the samples to be tested comprises:
observing the microstructures of the samples to be tested through a metalloscope; evenly intercepting five metallographic images for each sample to be tested; uniformly selecting 10 grains from each metallographic image to measure the diameter of ferrite grains according to a scale of 50 μm by using a scribbling tool in Image J software; and after all measurements, calculating an average value.

Optionally, the measurement process of the phase fractions comprises:
observing the microstructures of the samples to be tested through a metalloscope; evenly intercepting five metallographic images for each sample to be tested; counting an area proportion of ferrite grains in each metallographic image by using a Threshold function in Image J; characterizing phase fractions based on the area proportion; and after all measurements, calculating an average value.

Optionally, the process of testing the samples to be tested based on an improved U-shaped sensor probe comprises:
cutting the samples to be tested into samples with the size of 55×10×5 mm³ and placing in the improved U-shaped sensor probe respectively;
connecting a U-shaped sensor with a metal AC response real-time online measuring instrument and testing the samples.

Optionally, a manufacturing process of the improved U-shaped sensor probe comprises:
cutting out two grooves with a length of 10 mm, a width of 10 mm and a depth of 5 mm from the bottom of the U-shaped sensor by a metal processing technology, making the grooves close to an inner center position, and wrapping with enameled wires to obtain a sensor with one excitation coil and two induction coils.

Optionally, the test data comprises a real impedance part and an imaginary impedance part, and a process of constructing a frequency inductance curve based on the test data and the measurement results comprises:
transforming and calculating the test data to obtain an inductance value, obtaining a frequency value based on a logarithmic value, importing a measurement result after averaging five measurements of each sample into Origin, and drawing the frequency inductance curve.

Optionally, the response relational database characterizes response relationships of the grain size change and the phase fraction change of a single ferrite with electromagnetic signals.

The present invention has the following technical effects:

The present invention adopts an innovative electromagnetic characterization method to measure single microcomponents (grain size and phase fraction) in the steel and iron materials, which can be applied to the measurement of smaller samples, and is not limited to large steel plates. Compared with the traditional method, this innovative technology can improve measurement efficiency, reduce test time and resource cost, and expand the scope of application.

The samples in the experimental process of the present invention adopt an embedded measurement method to ensure that an induced magnetic field generated by the sensor can completely penetrate through the samples to be tested, thereby avoiding the phenomenon of magnetic leakage to the maximum extent. This design makes the measurement signal more intense and improves the accuracy of the measurement results. Accurate electromagnetic measurement data can be obtained through the multi-frequency electromagnetic characterization to further understand the influence of the grain size change and the phase fraction change of single ferrite on the electromagnetic signals under the multi-microcomponent structure of the steel and iron materials. Therefore, the method provides a feasible method for nondestructive characterization of the important microcomponents and performance of steel and iron by the electromagnetic technology, and is of great significance for material research, industrial application and academic research in related fields.

### DESCRIPTION OF DRAWINGS

Drawings forming a part of the present application are used for providing further understanding of the present application. Exemplary embodiments of the present application and the description are used for explaining the present application, but do not constitute an improper limitation to the present application. In the drawings:

FIG. 1 is a flow chart of a measurement method for characterizing ferrite grain size and phase fraction of duplex stainless steel in embodiments of the present invention;

FIG. 2 is a schematic diagram of multi-frequency electromagnetic characterization device connection and an induced magnetic field in embodiments of the present invention;

FIG. 3 is a heat treatment curve of industrial pure iron in embodiments of the present invention;

FIG. 4a is untreated industrial pure iron;

FIG. 4b is industrial pure iron held at 930° C. for 30 min;

FIG. 4c is industrial pure iron held at 930° C. for 60 min;

FIG. 4d is industrial pure iron held at 930° C. for 90 min;

FIG. 4e is industrial pure iron held at 930° C. for 120 min;

FIG. 5 is a corresponding frequency-inductance curve of a single ferrite structure with different grain sizes measured in embodiments of the present invention;

FIG. 6 is a heat treatment curve of 2205 duplex stainless steel in embodiments of the present invention;

FIG. 7a is held at 1050° C. for 30 min, then cooled with water, and then held at 850° C. for 120 min;

FIG. 7b is held at 1050° C. for 30 min, then cooled with water, and then held at 850° C. for 90 min;

FIG. 7c is held at 1050° C. for 30 min;

FIG. 7d is held at 1100° C. for 30 min;

FIG. 7e is held at 1250° C. for 30 min;

FIG. 8 is a corresponding frequency-inductance curve of different phase fractions (ferrite+austenite) measured in embodiments of the present invention;

FIG. 9a is the sensor probe;

FIG. 9b is the sample to be tested;

FIG. 10 is a schematic diagram of sensor testing in embodiments of the present invention.

### DETAILED DESCRIPTION

It should be explained that if there is no conflict, the embodiments in the present application and the features in the embodiments can be mutually combined. The present application will be described in detail below by reference to the drawings and in conjunction with the embodiments.

It should be noted that the steps shown in the flow chart of the figure can be executed in a computer system such as a set of computer-executable instructions, and although the logical sequence is shown in the flow chart, in some cases, the steps shown or described can be executed in a sequence different from the sequence here.

### Embodiment 1

The present embodiment provides a measurement method for characterizing ferrite grain size and phase fraction of duplex stainless steel.

A multi-frequency electromagnetic testing device is generally composed of two parts of an electromagnetic tester and a sensor. The electromagnetic tester is a main control unit of the multi-frequency electromagnetic testing device, and processes and analyzes the data by a built-in computer or processor. The electromagnetic tester can produce electromagnetic wave signals of different frequency ranges, and sends these signals to a tested object through the excitation coil. The sensor is an important part of the multi-frequency electromagnetic testing device, and composed of an excitation coil and an induction coil. The excitation coil is used for producing an electromagnetic field and introducing the electromagnetic wave signals into the tested object. The induction coil is responsible for receiving the response of the tested object to the electromagnetic wave signals and transmitting the signals to the electromagnetic tester for analysis and processing.

On the basis of the original multi-frequency electromagnetic testing probe, the bottom of the U-shaped sensor probe is processed in the patent of the present invention. Two grooves (for placing the samples to be tested) with a length of 10 mm, a width of 10 mm and a depth of 5 mm are cut out by a metal processing technology, and the grooves are close to an inner center position, and wrapped with enameled wires to manufacture a sensor with one excitation coil and two induction coils. The sensor in this design can effectively measure steel samples with different microstructures prepared by the heat treatment technology. The design of the grooves allows the samples to be easily and tightly placed on the sensor. The induced magnetic field generated inside the U-shaped ferrite can directly penetrate through the inside of the samples to be tested without magnetic leakage phenomenon, which can accurately reflect the changes in the microstructures of different samples.

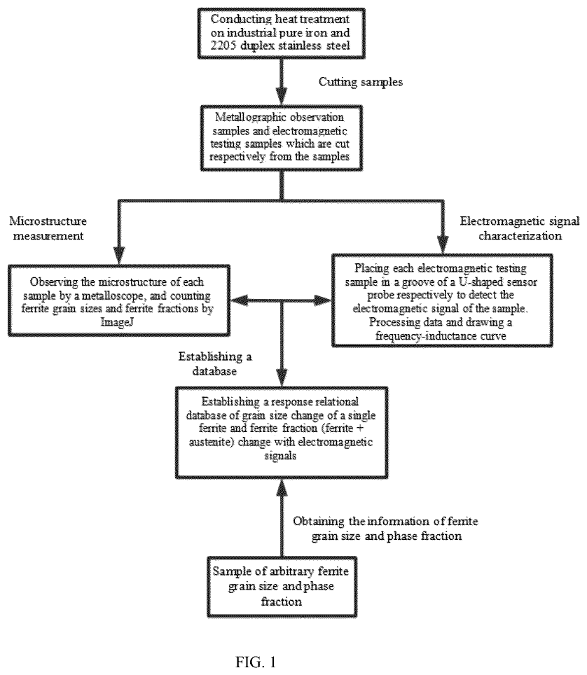

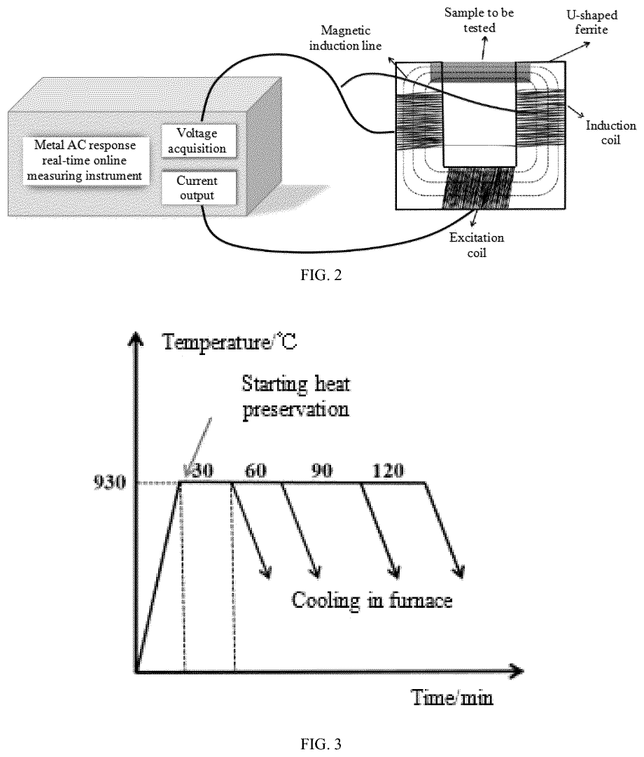

The specific implementation steps of the method in the patent are as follows (the method flow is shown in FIG. 1):

1. Heat treatment is conducted on industrial pure iron and 2205 duplex stainless steel through different heat preservation and cooling conditions. Single ferrite samples with different grain sizes and samples with different phase fractions (ferrite-austenite duplex stainless steel) are obtained respectively.

2. Wire cutting is conducted on the steel samples after heat treatment to obtain metallographic observation samples with the size of 10×10×5 mm³ and electromagnetic testing samples with the size of 55×10×5 mm³.

3. The microstructures of the samples are observed through a metalloscope; and the diameter of ferrite grains is measured in metallographic images according to a scale of 50 μm by using a scribbling tool in Image J software. Because the ferrite grain is geometrically roughly spherical, the diameter is used as a physical quantity to measure the grain size. When the phase fraction (ferrite+austenite) is measured, the area proportion of ferrite grains is counted by using the Threshold function in Image J. The area proportion is used to characterize the phase fraction, which can directly reflect the distribution of the phase on a two-dimensional plane. 5 metallographs are evenly captured for each observation sample by two measurement modes. 10 grains are evenly selected from each metallograph in measurement of the ferrite grain size to measure the diameter. An average value is calculated after measurement of the 5 metallographs. The proportion of ferrite area is counted for each metallograph in the measurement of the phase fraction. An average value is calculated after measurement of the 5 metallographs.

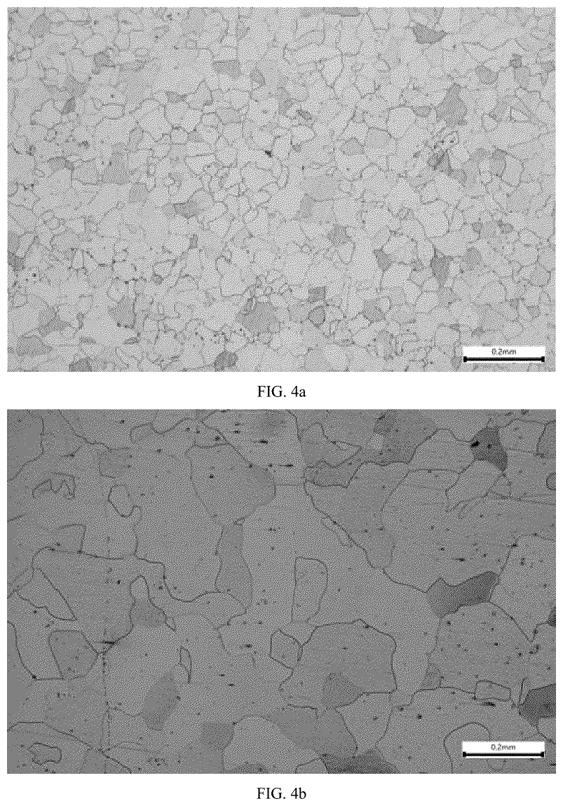

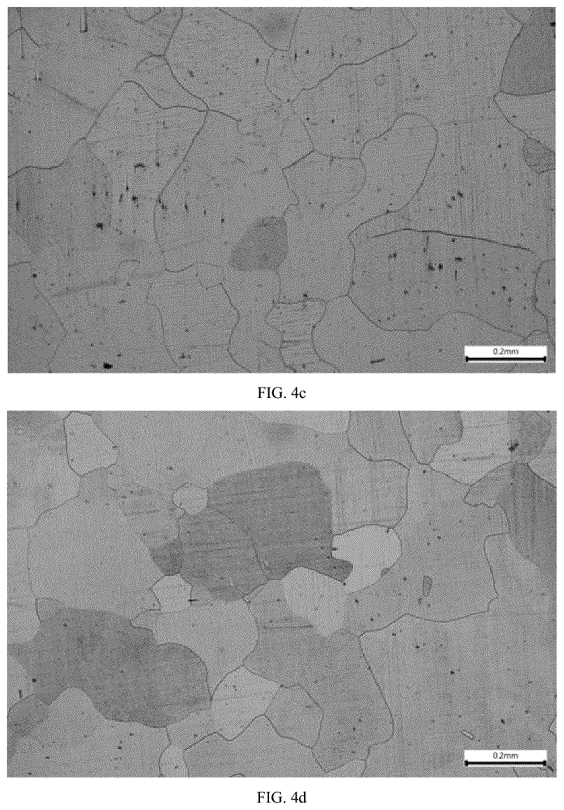

4. The prepared samples to be tested with different microstructures are cut into samples with the size of 55×10×5 mm³ and placed in the grooves of the U-shaped sensor probe respectively. The sensor is connected with a metal AC response real-time online measuring instrument. Firstly, the positive and negative poles of an AC signal output channel are clamped at both ends of a wire used to apply an excitation signal; a current acquisition channel is connected with a current sampling port of the AC signal output channel; and then the positive and negative electrode wires of a voltage acquisition channel are connected with the two induction coils respectively. The connection is shown in FIG. 3, and the sensor test is shown in FIGS. 4a-4e. In the measurement, the test current size is set to 600 mA, the measured value of pure iron at electromagnetic signal frequency is set to 1-100 Hz, and the measured value of 2205 duplex stainless steel is 1-25000 Hz.

5. The test data in the experiment comprises a real impedance part (Zre) and an imaginary impedance part (Zim). In the process of data processing, a real inductance value is calculated through the transformation of the formula (Zim/2πF, F is a frequency value), and the frequency in the result data is logarithmic. Five measured values of each sample are averaged and then imported into Origin, and a frequency-inductance curve is drawn. At the end of the experiment, a response relational database of grain size change of a single ferrite and phase fraction (ferrite+austenite) change with electromagnetic signals is summarized.

6. For any sample of ferrite grain size and phase fraction, contrast can be conducted with the response relational database established in step 5 according to the conclusion of the electromagnetic signals provided in step 4 of the method of the patent, to obtain the information of the ferrite grain size and the phase fraction.

### Embodiment 2

According to step 1 in embodiment 1, the industrial pure iron is firstly subjected to heat treatment respectively, and the conditions are shown in FIG. 3. After the treatment, each sample is cut according to step 2, and then the microstructure of the sample is observed and the grain size of the sample is measured according to the method of step 3. Part of the metallographs under each heat treatment condition are shown in FIGS. 4a-4e, and the measurement results of the grain sizes are shown in Table 1. Combined with the metallographs and the grain sizes, it can be seen that the samples of a single ferrite structure with different grain sizes are obtained from the heat treatment.

TABLE 1

| | Heat preservation time | | | | |
|---|---|---|---|---|---|
| | Untreated | 30 min | 60 min | 90 min | 120 min |
| Grain size/μm | 55 | 224 | 318 | 352 | 432 |

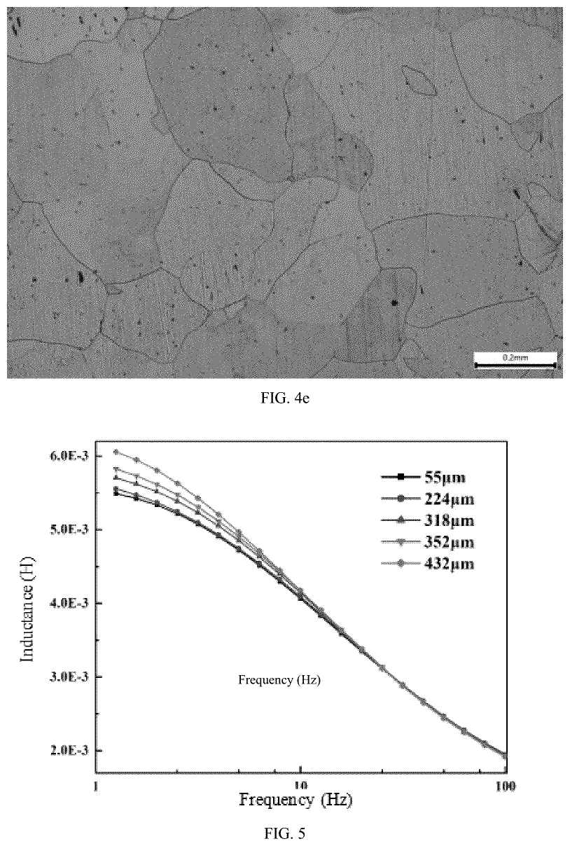

Subsequently, according to the method of step 4, the electromagnetic testing samples are prepared and an electromagnetic testing system is constructed. Electromagnetic signal testing is conducted for each sample. The measured data is processed according to the mode of step 5, and then a corresponding frequency-inductance curve is drawn combined with the ferrite grain size data in Table 1, as shown in FIG. 5. The change of the low-frequency inductance and the ferrite grain size can be observed, that is, the larger the ferrite grain size is, the higher the corresponding low-frequency inductance value is. A response relational database between the grain size change of a single ferrite and the electromagnetic signals is finally established.

### Embodiment 3

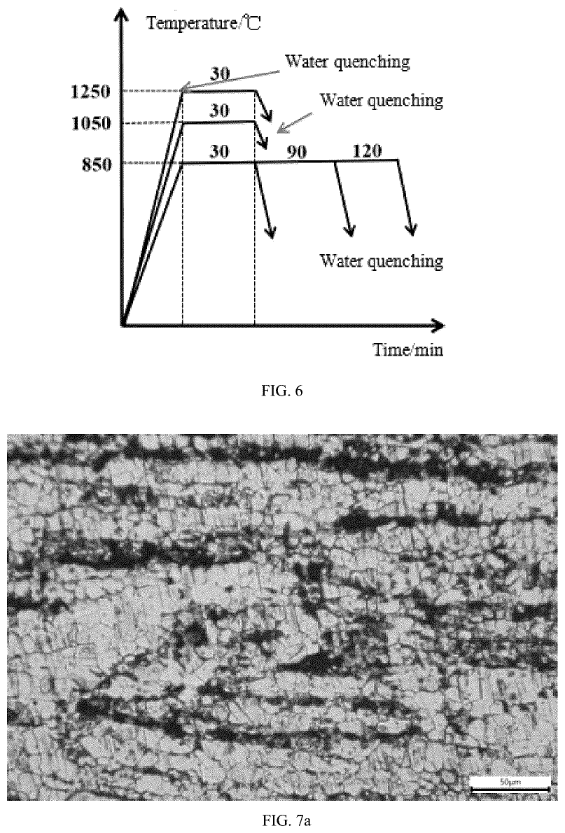

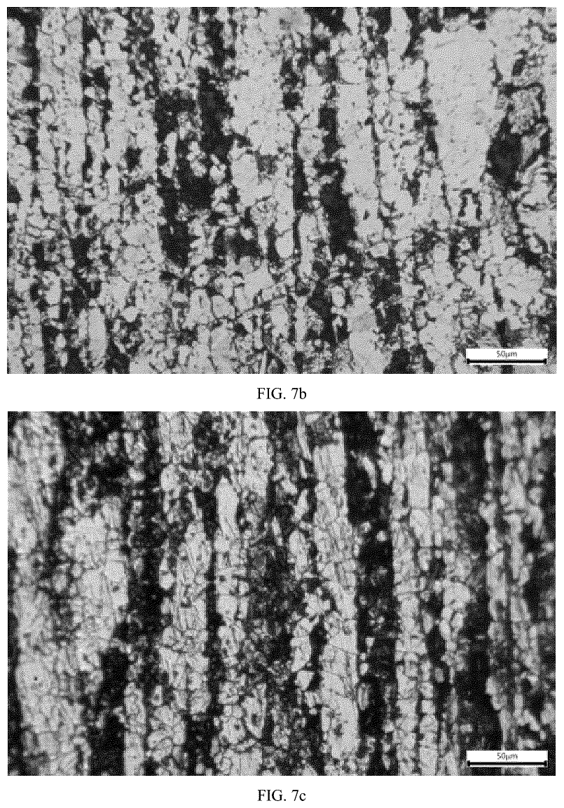

According to step 1 in embodiment 1, 2205 duplex stainless steel is firstly subjected to heat treatment respectively, and the conditions are shown in FIG. 6. After the treatment, each sample is cut according to step 2, and then the microstructure of the sample is observed and the phase fraction of the sample is measured according to the method of step 3. Part of the metallographs under each heat treatment condition are shown in FIGS. 7a-7e, and the measurement results of the phase fractions are shown in Table 2. Combined with the metallographs and the phase fractions, it can be seen that the samples with different phase fractions (ferrite+austenite) are obtained from the heat treatment.

TABLE 2

| | Heat Treatment Conditions | | | | |
|---|---|---|---|---|---|
| | Held at | Held at | | | |
| | 850° C. | 850° C. | | | |
| | for 120 min | for 90 min | Held at | Held at | Held at |
| | (after solid | (after solid | 1050° C. | 1100° C. | 1250° C. |
| | solution) | solution) | for 30 min | for 30 min | for 30 min |
| Phase fraction | 25 | 37 | 55 | 64 | 81 |
| (ferrite fraction)/% | | | | | |

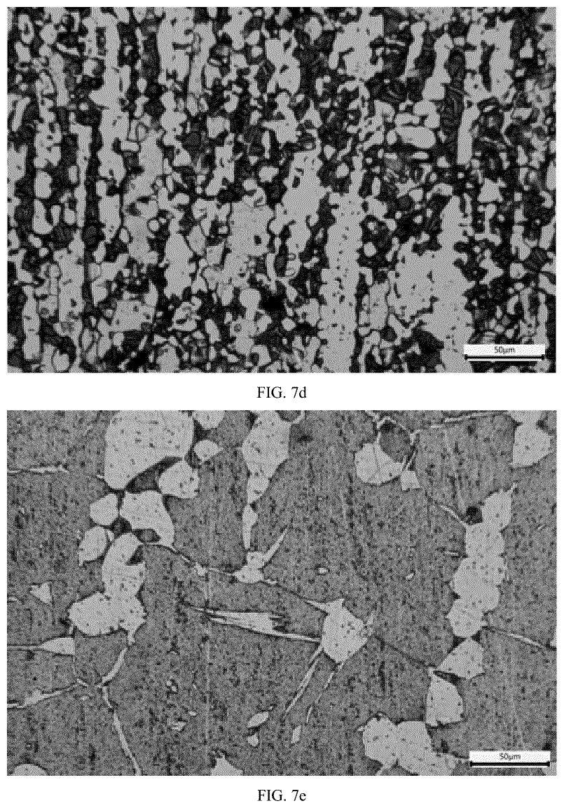

Subsequently, according to the method of step 4, the electromagnetic testing samples are prepared and an electromagnetic testing system is constructed. Electromagnetic signal testing is conducted for the samples with different phase fractions (ferrite+austenite). Then, measured data is processed according to the mode of step 5. A corresponding frequency-inductance curve is drawn combined with the phase fraction data in Table 2, as shown in FIG. 8. The change of the low-frequency inductance and the phase fractions (ferrite+austenite) can be observed, that is, the larger the ferrite (ferrite+austenite) fraction is, the higher the corresponding low-frequency inductance value is. A response relational database between the change of the phase fractions (ferrite+austenite) and the electromagnetic signals is finally established.

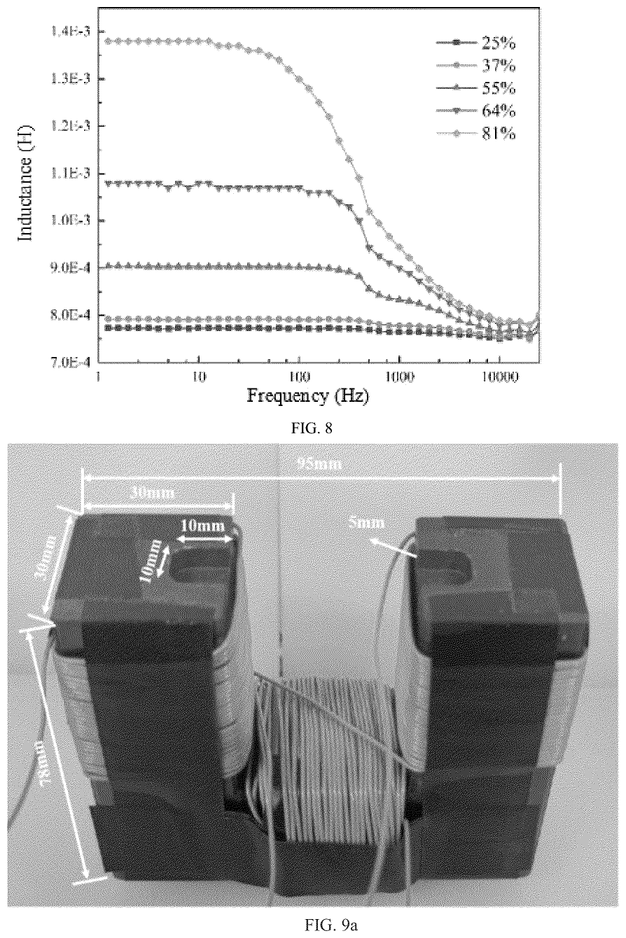

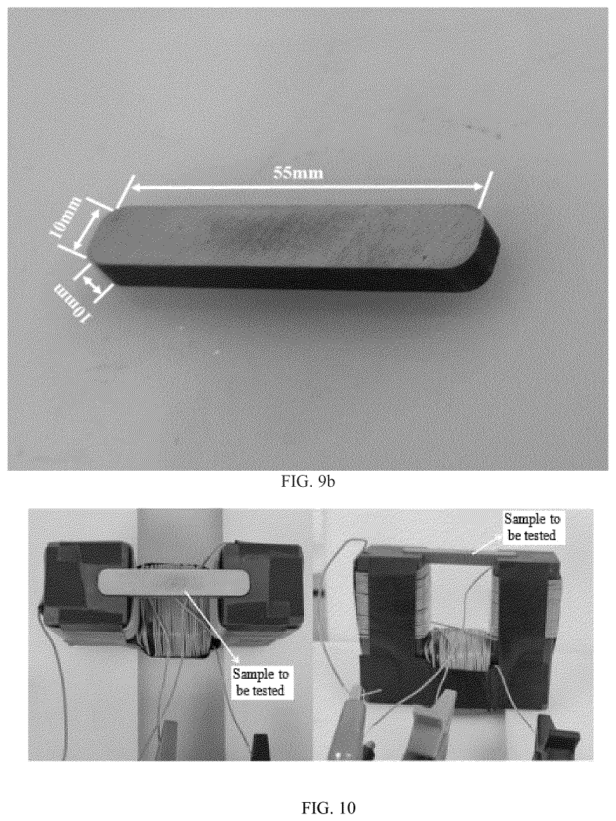

The schematic diagram of the samples to be tested is shown in FIG. 9b and the self-made sensor probe in the technical solution of the present invention is shown in FIG. 9a, and the schematic diagram of the testing process is shown in FIG. 10. The present invention adopts the heat treatment mode to prepare single ferrite microcomponent samples with different grain sizes and duplex stainless steel samples with different phase fractions. Electromagnetic signal measurement is conducted on the samples through the multi-frequency electromagnetic sensor probe prepared by the patent of the present invention; the corresponding relationship between the microcomponents and the electromagnetic signals is clarified; the response mechanism of the corresponding electromagnetic testing is revealed; and the ferrite grain sizes and phase fractions are characterized.

Through this improved design, the electromagnetic signal difference caused by the change of the grain size and the phase fraction of a single ferrite can be accurately measured, which provides important experimental data and theoretical support for the research and application of electromagnetic characterization of steel microstructure.

The above only describes preferred specific embodiments of the present application, but the protection scope of the present application is not limited thereto. Any change or replacement contemplated easily by those skilled in the art familiar with the technical field within the technical scope disclosed by the present application shall be covered within the protection scope of the present application. Therefore, the protection scope of the present application should be determined by the protection scope of the claims.

1. A measurement method for characterizing ferrite grain size and phase fraction of duplex stainless steel, comprising the following steps:
setting heat preservation and cooling conditions respectively, and conducting heat treatment on industrial pure iron and duplex stainless steel respectively to obtain samples with different grain sizes and different phase fractions;
conducting wire cutting on the samples after heat treatment to obtain the samples to be tested;
measuring ferrite grain diameters and phase fractions of the samples to be tested to obtain measurement results;
testing the samples to be tested based on an improved U-shaped sensor probe to obtain test data;
constructing a frequency inductance curve based on the test data and the measurement results; and constructing a response relational database based on the frequency inductance curve;
obtaining the information of the sample of arbitrary ferrite grain size and phase fraction based on the response relational database.

2. The measurement method for characterizing ferrite grain size and phase fraction of duplex stainless steel according to claim 1, wherein the samples to be tested comprise metallographic observation samples with a size of 10×10×5 mm³ and electromagnetic testing samples with a size of 55×10×5 mm³.

3. The measurement method for characterizing ferrite grain size and phase fraction of duplex stainless steel according to claim 1, wherein the process of measuring the ferrite grain diameters of the samples to be tested comprises:
observing the microstructures of the samples to be tested through a metalloscope; evenly intercepting five metallographic images for each sample to be tested; uniformly selecting 10 grains from each metallographic image to measure the diameter of ferrite grains according to a scale of 50 μm by using a scribbling tool in Image J software; and after all measurements, calculating an average value.

4. The measurement method for characterizing ferrite grain size and phase fraction of duplex stainless steel according to claim 1, wherein the measurement process of the phase fractions comprises:
observing the microstructures of the samples to be tested through a metalloscope; evenly intercepting five metallographic images for each sample to be tested; counting an area proportion of ferrite grains in each metallographic image by using a Threshold function in Image J; characterizing phase fractions based on the area proportion; and after all measurements, calculating an average value.

5. The measurement method for characterizing ferrite grain size and phase fraction of duplex stainless steel according to claim 1, wherein the process of testing the samples to be tested based on an improved U-shaped sensor probe comprises:
cutting the samples to be tested into samples with a size of 55×10×5 mm³ and placing in the improved U-shaped sensor probe respectively;
connecting a U-shaped sensor with a metal AC response real-time online measuring instrument and testing the samples.

6. The measurement method for characterizing ferrite grain size and phase fraction of duplex stainless steel according to claim 1, wherein a manufacturing process of the improved U-shaped sensor probe comprises:
cutting out two grooves with a length of 10 mm, a width of 10 mm and a depth of 5 mm from the bottom of the U-shaped sensor by a metal processing technology, making the grooves close to an inner center position, and wrapping with enameled wires to obtain a sensor with one excitation coil and two induction coils.

7. The measurement method for characterizing ferrite grain size and phase fraction of duplex stainless steel according to claim 1, wherein the test data comprises a real impedance part and an imaginary impedance part, and a process of constructing a frequency inductance curve based on the test data and the measurement results comprises:
transforming and calculating the test data to obtain an inductance value, obtaining a frequency value based on a logarithmic value, importing a measurement result after averaging five measurements of each sample into Origin, and drawing the frequency inductance curve.

8. The measurement method for characterizing ferrite grain size and phase fraction of duplex stainless steel according to claim 1, wherein the response relational database characterizes response relationships of the grain size change and the phase fraction change of a single ferrite with electromagnetic signals.
